(19) United States
(12) Patent Application Publication
(10) Pub. No.: US 2023/0247992 A1
(43) Pub. Date: Aug. 10, 2023

(54) ARTHROPOD CONTROL COMPOSITION

(71) Applicant: Firmenich SA, Satigny (CH)

(72) Inventor: Vincent HARRACA, Satigny (CH)

(21) Appl. No.: 18/004,300

(22) PCT Filed: Jul. 13, 2021
(86) PCT No.: PCT/EP2021/069430

§ 371 Date: Jan. 4, 2023

(30) Foreign Application Priority Data
Jul. 15, 2020 (EP) 20185988.1

(51) Int. Cl. A01N 37/18; A01P 17/00; A01N 37/02; A01N 35/04; A01N 31/16; A01N 65/22; A01N 43/12

(52) U.S. Cl. CPC A01N 37/18; A01N 31/16; A01N 35/04; A01N 37/02; A01N 43/12; A01N 65/22; A01P 17/00

(57) ABSTRACT

The present invention relates to an arthropod control composition, methods and uses to control arthropods as well as arthropod control articles comprising the same.

### TECHNICAL FIELD

The present invention relates to an arthropod control composition, methods and uses to control arthropods as well as arthropod controlling articles comprising the same.

### BACKGROUND

Many mammals, including humans, are suffering from the action of arthropods. Some arthropods, such as for example mosquitoes and ticks, are not desirable for vertebrates such as mammals and in particular human subjects as they bite and, consequently, cause itching, transmission of diseases and/or germs or may be the cause for other diseases and/or conditions.

Arthropod control compositions include active substances and when applied to skin, clothing, or other surfaces, they may discourage arthropods from landing or climbing on that surface. Arthropod control agents help preventing and controlling the outbreak of arthropod-borne diseases, such as malaria, etc.

Some of the known arthropod controlling agents and composition, however, have certain drawbacks as they can have negative effects, i.e. negative olfactive properties, such as no or bad smell, or in turn only weak arthropod controlling, in particular arthropod repelling properties.

There is a need to provide arthropod control compositions that have both good olfactive properties, i.e. a good hedonic effect, and good arthropod controlling, in particular arthropod repelling properties.

The prior art does not disclose or suggest the arthropod control compositions according to the present invention.

### FIGURE LEGENDS

FIG. 1: number of mosquitoes landing within 2 minutes on the warm body treated with different substances at different concentrations. Test with ethanol only (control) are displayed as 0 mg/m.

FIG. 2: number of mosquitoes landing within 2 minutes on the warm body while the air of the cage was treated with different substances at different concentrations. Test with solvent only (control) are displayed as 0 µg/L of air and n.d. stands for no data.

FIG. 3: percentage of repellence in the arm-in-cage test. Each bar represents the mean repellence ±SD on 3 volunteers and n.d. stands for no data.

### DETAILED DESCRIPTION

The present invention relates to an arthropod, preferably insect, control composition comprising one or more substance(s), wherein the substance is selected from the group consisting of: (E)-1-(2,6,6-trimethylcyclohex-2-en-1-yl)but-2-en-1-one, (3Z)-3-butylidene-2-benzofuran-1-one, 4-ethenyl-2-methoxyphenol, Cognac oil green, Labdanum extract (Cistus spp.), 5-pentyloxolan-2-one, chromen-2-one, (2E)-3,7-dimethylocta-2,6-dienal, 4-hydroxy-3-methoxybenzaldehyde, (5R)-2-methyl-5-prop-1-en-2-ylcyclohex-2-en-1-one, Mentha spicata oil, 6-hexyloxan-2-one, 5-methyl-2-propan-2-ylcyclohexyl] acetate, Nigella damascena oil, 2-phenylethanol, 6-pentyloxan-2-one, (4-methoxyphenyl)methyl acetate, Syzygium aromaticum oil, 3,4,4a,5,6,7,8,8a-octahydrochromen-2-one, (1S,6R)-3,7,7-trimethylbicyclo[4.1.0]hept-3-ene, 2-phenylethyl 2-methylpropanoate, methyl 2-(3-oxo-2-pent-2-enylcyclopentyl)acetate, (4R)-4-(2-methoxypropan-2-yl)-1-methylcyclohexene, Mentha piperita oil, 2-methoxy-4-[(E)-prop-1-enyl]phenol, 2-methyl-3-(4-propan-2-ylphenyl)propanal and (4-methoxyphenyl)methanol.

The substances in the composition of the invention are known in the art and can be readily synthesized or obtained. The following list provides the CAS number for each of the substances: 5-pentyloxolan-2-one (CAS number 104-61-0), chromen-2-one (CAS number 91-64-5), 2-methyl-3-(4-propan-2-ylphenyl)propanal (CAS number 103-95-7), (4-methoxyphenyl)methanol (CAS number 105-13-5), (2E)-3,7-dimethylocta-2,6-dienal (CAS number 5392-40-5), 4-hydroxy-3-methoxybenzaldehyde (CAS number 121-33-5), (5R)-2-methyl-5-prop-1-en-2-ylcyclohex-2-en-1-one (CAS number 6485-40-1), 4-ethenyl-2-methoxyphenol (CAS number 7786-61-0), Labdanum extract (Cistus spp.) (CAS number 84775-64-4 / 8016-26-0), Cognac oil green (CAS number 8016-21-5), (3Z)-3-butylidene-2-benzofuran-1-one (CAS number 551-08-6), (E)-1-(2,6,6-trimethylcyclohex-2-en-1-yl)but-2-en-1-one (CAS number 24720-09-0), 6-hexyloxan-2-one (CAS number 710-04-3), 5-methyl-2-propan-2-ylcyclohexyl] acetate (CAS number 2623-23-6), Nigella damascena oil (CAS number 73507-35-4 / 90064-31-6), 2-phenylethanol (CAS number 60-12-8), 6-pentyloxan-2-one (CAS number 705-86-2), (4-methoxyphenyl)methyl acetate (CAS number 104-21-2), 3,4,4a,5,6,7,8,8a-octahydrochromen-2-one (CAS number 4430-31-3), (1S,6R)-3,7,7-trimethylbicyclo[4.1.0]hept-3-ene (CAS number 498-15-7), 2-phenylethyl 2-methylpropanoate (CAS number 103-48-0), methyl 2-(3-oxo-2-pent-2-enylcyclopentyl)acetate (CAS number 20073-13-6), (4R)-4-(2-methoxypropan-2-yl)-1-methylcyclohexene (CAS number 30199-25-8), 2-methoxy-4-[(E)-prop-1-enyl]phenol (CAS number 5932-68-3), Mentha spicata oil (CAS number 8008-79-5 / 84696-51-5), Syzygium aromaticum oil (CAS number 8000-34-8 / 68917-29-3) and Mentha piperita oil (CAS number 8006-90-4 / 84082-70-2).

In a particular embodiment, the substance used for repellence, more preferably mosquito repellence, is selected from Labdanum extract, Cognac oil green, (3Z)-3-butylidene-2-benzofuran-1-one, 2-methyl-3-(4-propan-2-ylphenyl)propanal, methyl 2-(3-oxo-2-pent-2-enylcyclopentyl)acetate, Nigella damascena oil, (2E)-3,7-dimethylocta-2,6-dienal, 5-pentyloxolan-2-one, Syzygium aromaticum oil, 4-ethenyl-2-methoxyphenol, 4-hydroxy-3-methoxybenzaldehyde, 6-pentyloxan-2-one, (1S,6R)-3,7,7-trimethylbicyclo[4.1.0]hept-3-ene, chromen-2-one, 6-hexyloxan-2-one, 2-methoxy-4-[(E)-prop-1-enyl]phenol, 5-methyl-2-propan-2-ylcyclohexyl] acetate, (4-methoxyphenyl)methanol.

In a particular embodiment, the substance used for repellence, more preferably mosquito repellence, is selected from (3Z)-3-butylidene-2-benzofuran-1-one, methyl 2-(3-oxo-2-pent-2-enylcyclopentyl)acetate, Nigella damascena oil, (2E)-3,7-dimethylocta-2,6-dienal, Syzygium aromaticum oil, 6-pentyloxan-2-one, chromen-2-one, 2-phenylethyl 2-methylpropanoate, 4-ethenyl-2-methoxyphenol.

In a particular embodiment, the substance used for spatial repellence, more preferably mosquito spatial repellence, is selected from Cognac oil green, (E)-1-(2,6,6-trimethylcyclohex-2-en-1-yl)but-2-en-1-one, 2-methyl-3-(4-propan-2-ylphenyl)propanal, 4-ethenyl-2-methoxyphenol, methyl 2-(3-oxo-2-pent-2-enylcyclopentyl)acetate, 6-hexyloxan-2-one, chromen-2-one, 6-pentyloxan-2-one, Mentha piperita oil, 4-hydroxy-3-methoxybenzaldehyde, Syzygium aromaticum oil, 5-pentyloxolan-2-one, 2-phenylethanol, Nigella damascena oil, Mentha spicata oil, (5R)-2-methyl-5-prop-1-en-2-ylcyclohex-2-en-1-one, 2-methoxy-4-[(E)-prop-1-enyl]phenol, (4-methoxyphenyl)methanol, (4R)-4-(2-methoxypropan-2-yl)-1-methylcyclohexene, 2-methoxy-4-[(E)-prop-1-enyl]phenol, (4-methoxyphenyl)methyl acetate.

In a particular embodiment, the substance used for spatial repellence, more preferably mosquito repellence, is selected from 4-ethenyl-2-methoxyphenol, methyl 2-(3-oxo-2-pent-2-enylcyclopentyl)acetate, chromen-2-one, 6-pentyloxan-2-one, Mentha piperita oil, 4-hydroxy-3-methoxybenzaldehyde.

In a particular embodiment, the substance used for deterrence, more preferably tick deterrence, is selected from (E)-1-(2,6,6-trimethylcyclohex-2-en-1-yl)but-2-en-1-one, 4-ethenyl-2-methoxyphenol, (3Z)-3-butylidene-2-benzofuran-1-one, 5-pentyloxolan-2-one, chromen-2-one, 2-methoxy-4-[(E)-prop-1-enyl]phenol, 2-methyl-3-(4-propan-2-ylphenyl)propanal.

In a particular embodiment, the substance used for deterrence, more preferably tick deterrence, is selected from 4-ethenyl-2-methoxyphenol, (3Z)-3-butylidene-2-benzofuran-1-one, 5-pentyloxolan-2-one, 2-methoxy-4-[(E)-prop-1-enyl]phenol.

In a particular embodiment, the arthropod control composition comprises the compound or composition in an amount of 0.01 to 90 wt.%, more preferably 0.2 to 30 wt.%, based on the total weight of the composition.

In a preferred embodiment of the invention the arthropod control composition has a good hedonic profile.

The present inventors have good appreciated that it may be desirable for arthropod control compositions to have an acceptable hedonic profile. This is because the compositions may be used in close proximity to a consumer and, as can be understood, any an unpleasant hedonic profile may inhibit the use of the composition.

The term “arthropod” has the normal meaning for a skilled person in the technical field. Arthropods include invertebrate animals, such as insects, arachnids, and crustaceans, that have a segmented body and jointed appendages. Arthropods usually have a chitinous exoskeleton molted at intervals, and a dorsal anterior brain connected to a ventral chain of ganglia.

Arthropods in the present invention’s understanding relate to undesired arthropods, meaning that their presence in the air, on the surface of an article, the surface of a plant or the surface of a vertebrate, such as a human subject or other mammal, preferably human subject, is not desired. Preferably undesired arthropods are pest arthropods that impact plants and animals, e.g. thrips, aphids, beetles, moth, mealybug, scale etc., more preferably pest arthropods that impact animals, e.g. ants, termites, cockroaches, flies, etc., even more preferably blood feeding arthropods that impact vertebrates, e.g. biting fly, bed bug, kissing bug, flea, lice, mosquitos and ticks, even more preferably mosquitos and ticks.

The reason why the presence of an arthropod is not desired might be that the arthropod’s presence in the air is unpleasant to a subject, the contact of an arthropod on an article transfers diseases and/or germs or the arthropod bites an organism and causes itching, the transmission of diseases and/or germs or the arthropod feeding may be the cause for other diseases and/or conditions.

In a particular embodiment, the arthropod is an insect or an arachnid, preferably an insect.

The term “insect” has the normal understanding by a skilled person the technical field. An insect is described by a well-defined head, thorax, and abdomen, only three pairs of legs, and typically one or two pairs of wings.

In a particular embodiment, the insect is a mosquito, biting fly, bedbug, kissing bug, flea, lice, ant, termite, cockroach, fly, aphid, beetle, thrips, moth, mealybug or scale bug, more preferably a mosquito.

The term “arachnid” has the normal understanding by a skilled person the technical field. An arachnid is described having a segmented body divided into two regions of which the anterior bears four pairs of legs but no antennae.

In a particular embodiment, the arachnid is a tick, mite, chigger or spider, more preferably a tick.

The expression “control”, “arthropod control”, “insect control” or “arachnid control” or the like has the normal meaning for a skilled person in the technical field.

“Controlling” in the context of the present invention defines the ability of an arthropod controlling composition according to the present invention to attract, deter, kill or repel an arthropod, preferably deter or repel an arthropod and even more preferably repel an arthropod.

“Attracting” according to the present invention defines the ability of an arthropod attractant composition according to the invention to increase or encourage contact or the presence of an arthropod at the arthropod attractant source, such as in the air, on the surface of an article or on the surface of an vertebrate, such as a human subject or other mammal, preferably an article such as a trapping device, the arthropod attractant compound or composition has been applied to.

“Repellency” according to the present invention defines the ability of an arthropod repellent composition according to the present invention to minimize, reduce, discourage or prevent approach or the presence of an arthropod at the arthropod repellent source, such as in the air, on the surface of an article or on the surface of an vertebrate, such as a human subject or other mammal, preferably human subject, to which the arthropod repellent compound or composition has been applied to.

“Deterring” according to the present invention defines the ability of an arthropod deterrent composition according to the invention to minimize, reduce, discourage or prevent contact or the presence of an arthropod at the arthropod deterrent source, such as in the air, on the surface of an article or on the surface of an vertebrate, such as a human subject or other mammal, preferably human subject, to which the arthropod deterrent compound or composition has been applied to. Typically, the deterrent effect is shown when used as feeding deterrent hindering a pest from subsequent food intake or oviposition or physical contact after an initial tasting of the arthropod deterrent compound or composition.

“Spatial Repellency” according to the present invention defines the ability of an arthropod repellent composition according to the present invention to minimize, reduce, discourage or prevent approach or the presence of an arthropod at the arthropod repellent source, such as in the air, on the surface of an article or on the surface of an vertebrate, such as a human subject or other mammal, preferably human subject, to which the arthropod repellent compound or composition has been applied to. Typically, the spatial repellency effect is shown when spatial repellent compound or composition released, sprayed, spread or diffused in the air or liquid hinder a pest from entering the zone in which the spatial repellent compound or composition is present. Repellence occurs therefore from a distance, the pest not necessarily entering in direct contact with the treated article or organism to protect.

“Killing” according to the present invention defines the ability of arthropod killing composition according to the present invention to kill an arthropod at the arthropod killing source, such as in the air, on the surface of an article or on the surface of an vertebrate, such as a human subject or other mammal, preferably human subject, to which the arthropod killing compound or composition has been applied to. When an arthropod killing composition is applied to a plant, an animal or human subject, it is applied in an amount which is killing to the arthropod but not to the subject.

In a particular embodiment, the arthropod control composition is an arthropod repelling composition, preferably an insect repelling composition, more preferably a mosquito repelling composition.

In a particular embodiment, the arthropod controlling source is the surface and/or the air in the vicinity of an article, preferably a candle, coil, electric diffuser, wristband, patch, collar, ear tag, clothes, fabrics, papers, biochar, cardboard, cellulosic pads, bed nets, screen, curtains, furniture, walls, ground or paint, or the surface of a subject, preferably the surface of a vertebrate, such as a human subject or other mammal, preferably human subject, i.e. the skin of a human subject treated with a product such as spray, aerosol, cream, roll on, wristband, lotion, soap, shampoo, sunscreen or patch or a cloth treated with a product such as laundry powder, liquid detergent, spray, lotion, powder.

The arthropod controlling effect according to the present invention is determined on mosquitoes using an adapted Warm Body assay as defined in Kröber T, Kessler S, Frei J, Bourquin M, Guerin PM. An in vitro assay for testing mosquito controlling compounds employing a warm body and carbon dioxide as a behavioral activator. J Am Mosq Control Assoc. 2010; 26:381-386. Further information is provided in the accompanying examples.

The controlling effect, repellence & spatial repellence, according to the present invention is determined by testing the Warm Body assay against the yellow fever mosquito, Aedes aegypti Rockefeller strain. A. aegypti is a model organism for controlling tests and one of the recommended model organisms by the World Health Organization (WHO) as it is a very aggressive, anthropophilic mosquito species that shows generally low sensitivity to arthropod controlling compounds. Observations of controlling efficacy were made on host-seeking females of uniform age, 5 to 10 days old selected as mentioned in the publication mentioned hereinabove. Tested hungry females had access to 10% sugar solution but were not blood-fed. Further information is provided in the accompanying examples.

The published protocol has been adapted in not manually counting the landing mosquitoes but automatically using an automatic counting software, the switch from Anopheles gambiae to A. aegypti led to a decrease of mosquitoes’ number placed in the tested cage due to the size difference (i.e. 30 mosquitoes instead of 50) and to an increase of lighting as A. aegypti is a diurnal mosquitoes (i.e. 150 lux instead of 4 lux). Further information is provided in the accompanying examples.,

The controlling effect, repellence & spatial repellence, according to the present invention is also determined according to an arm in the box method adapted from the WHO Guidelines for efficacy testing of mosquito repellents for human skin (WHO/CDS/NTD/WHOPES/2009.4). The readiness of 100 hungry female mosquitoes A. aegypti to a test substance is assessed by comparing the results of an untreated arm to a treated in when inserted into the cage (40×40×40 cm) for 30 seconds (negative control) three times. Further information is provided in the accompanying examples.

The activity for substances to repel arachnids such as ticks was assessed using the protocol of the in-vitro Warm Plate Assay as defined in Kröber T, Bourquin M, Guerin PM. 2013. A standardized in vivo and in vitro test method for evaluating tick repellents. Pestic. Biochem. Phys. 107(2):160-168. Further information is provided in the accompanying examples.

In a particular embodiment, the amount and selection of the substance is in a way that it contributes, enhances or improves both, the arthropod control activity and the hedonic character of the composition.

In one embodiment, the arthropod control composition may further comprise an arthropod control co-ingredient. By “arthropod control co-ingredient” is understood an ingredient capable of imparting additional arthropod controlling benefits to the arthropod controlling effect of the composition herein described.

In one embodiment, the substance herein described is capable to modify, enhance or improve the arthropod controlling effect of the arthropod control co-ingredient, e.g. by reducing the amount of the arthropod control co-ingredient within a composition. This can be particularly beneficial in case the arthropod control co-ingredient is harmful to human subjects at a certain dose or in case the arthropod control co-ingredient has negative olfactive properties at a certain dose.

According to a particular embodiment, the combination of the substance herein described and an arthropod control co-ingredient results in a synergistic arthropod controlling effect.

According to a particular embodiment, the combination of substance herein described and an arthropod control co-ingredient results in a modified, pleasant, enhanced or improved olfactory impression of the overall composition in comparison to its single ingredients.

According to one embodiment arthropod control co-ingredient is selected from the group consisting of: N,N-diethyl-3-methylbenzamide (DEET), ethyl butylacetylaminopropionate (IR3535); para-menthan-3,8-diol (PMD); 1-(1-methylpropoxycarbonyl)-2-(2-hydroxaethyl)piperidin (Icaridin); Cedarwood oil (China), Cedarwood oil (Texas), Cedarwood oil (Virginia), Cinnamon oil, Citronella oil, Cornmint oil, Cymbopogon winterianus oil fractionated hydrated cyclized, decanoic acid, Eucalyptus citriodora oil Eucalyptus citriodora oil hydrated cyclized, eugenol, Garlic oil, geraniol, Geranium oil, Lavender, Lavandula hybrida ext., Lavandin oil, Lemon oil, Lemongrass oil, Margosa extract, Metofluthrin, mixture of cis- and trans-p-menthane-3,8 diol, N,N-diethyl-meta-toluamide, nonanoic acid, Rosemary oil, Thyme oil, Wintergreen oil, 2,3,4,5-bis(butyl-2-ene)tetrahydrofurfural (MGK Repellent 11), cineole, cinnamaldehyde, citronellal, citronellol,, coumarin, dibutyl phthalate, diethyl phthalate, dimethyl anthranilate, dimethyl phthalate, ethyl vanillin, Eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, Lime oil, limonene, linalool, methyl anthranilate, Mint oil, myrcene, Neem oil, sabinene, β-caryophyllene, (1H-indol-2-yl)acetic acid, anethole, Anise oil, Basil oil, Bay oil, camphor, ethyl salicylate, Evergreen oils (pine oil), (1,3,4,5,6,7-hexahydro-1,3-dioxo-2H-isoindol-2-yl)methyl (1R-trans)-2,2-dimethyl-3-(2-methylprop-1-enyl)cyclopropanecarboxylate (d-Tetramethrin), (RS)-3-Allyl-2-methyl-4-oxocyclopent-2-enyl-(1R,3R;1R,3S)-2,2-dimethyl-3-(2- methylprop-1-enyl)-cyclopropanecarboxy late (mixture of 4 isomers 1R trans, 1R:1R trans, 1S: 1R cis, 1R: 1R cis,1S 4:4:1:1) (d-Allethrin), (RS)-α-cyano-3phenoxybenzyl-(1RS)-cis, trans-3-(2,2-dichlorovinyl)-2,2-dimethylcyclopropanecarboxylate (Cypermethrin), 2-methyl-4-oxo-3-(prop-2-ynyl)cyclopent-2-en-1-yl 2,2-dimethyl-3-(2-methylprop-1-enyl)cyclopropanecarboxylate (Prallethrin), Acetamiprid, Azadirachtin, Bendiocarb, Bifenthrin, boric acid, Chlorpyrifos, Deltamethrin, Diazinon, Dichlorvos, eugenol, Fipronil, Imidacloprid, linalool, Malathion, Maltodextrin, Metofluthrin, Nicotine, Permethrin, Pyrethrins and Pyrethroids, Rotenone, silicium dioxide (Kieselguhr), S-Methoprene, Spinosad (Spinosyn A), Spinosyn D, Tetramethrin, Transfluthrin and mixtures thereof.

In a particular embodiment, the arthropod control co-ingredient is comprised in an amount of from 0.02 to 80 wt.%, more preferably in an amount of from 0.05 to 70 wt.%, even more preferably in an amount of from 0.1 to 60 wt.%, based on the total weight of the composition. Thereby, it is understood that the composition comprises the arthropod control co-ingredient in a minimum amount of at least 0.2 wt.%, at least 0.05 wt.% or at least 0.1 wt.% and a maximum amount of not more than 80 wt.%, not more than 70 wt.% or not more than 60 wt.%, based on the total weight of the composition.

In a particular embodiment, within the limitations of the amount of the substance in the composition as stated above, the substance in the composition of the invention and the arthropod control co-ingredient are comprised in the composition in a weight range of 90:10 to 10:90, preferably in a weight range of 80:20 to 20:80, more preferably in a weight range of 65:35 to 35:65 and most preferably in a weight range of 60:40 to 40:60. It is herein also understood that substance and the arthropod control co-ingredient can be comprised in the composition in any weight range combination as mentioned herein-before, such as 90:10 to 20:80, preferably 35:65 and more preferably 40:60, 80:20 to 10:90, preferably 35:65 and more preferably 40:60, 65:35 to 10:90, preferably 20:80 and more preferably 40:60 or 40:60 to 10:90, preferably 20:80 and more preferably 35:65.

In one embodiment, the arthropod control composition may further comprise perfume ingredients. Perfume ingredients are understood as contributing, modifying, enhancing or improving the olfactory character of the composition but does not contribute to, enhance or improve the arthropod controlling effect of the composition.

The arthropod control composition can further comprise a carrier. By “carrier” is understood a material with which the active compound is mixed or formulated to facilitate its application a locus or other object to be treated, or its storage, transport and/or handling. Said carrier may be of inorganic or organic or of synthetic natural origin. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly cannot be exhaustive. However, one can cite as non-limiting examples, solvents such as butylene or propylene glycol, glycerol, dipropylene glycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethanol, tri-ethyl citrate, 2-methylprop-1-ene and 2-(2-ethoxyethoxy)ethanol or mixtures thereof, particular suitable are dipropylene glycol, 2-methylprop-1-ene and 2-(2-ethoxyethoxy)ethanol and mixtures thereof.

For the compositions which comprise both a carrier, other suitable carriers than those previously specified, can be also ethanol, water/ethanol mixtures, limonene or other terpenes, isoparaffins such as those known under the trademark Isopar (origin: Exxon Chemical) or glycol ethers and glycol ether esters such as those known under the trademark Dowanol (origin: Dow Chemical Company), or hydrogenated castors oils such as those known under the trademark Cremophor RH 40 (origin: BASF).

Solid carrier is meant to designate a material to which the arthropod control composition or some element of the arthropod control composition can be chemically or physically bound. In general, such solid carriers are employed either to stabilize the composition, or to control the rate of evaporation of the compositions or of some ingredients. The use of solid carrier is of current use in the art and a person skilled in the art knows how to reach the desired effect. However, by way of non-limiting example of solid carriers, one may cite absorbing gums or polymers or inorganic material, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs- und Geliermittel in Lebensmitteln, Band 2 der Schriftenreihe Lebensmittelchemie, Lebensmittelqualität, Behr’s Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation technique.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Others resins one are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate.

Some of the seminal literature related to the encapsulation by polycondensation of amino resins, namely melamine-based resins with aldehydes includes represented by articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. US 4′396′670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinency, which disclose suitable uses of such microcapsules, are represented for example by the article of H.Y.Lee et al. Journal of Microencapsulation, 2002, vol. 19, pages 559-569, international patent publication WO 01/41915 or yet the article of S. Bône et al. Chimia, 2011, vol. 65, pages 177-181.

The present invention also relates to a method for arthropod, preferably insect, control which comprises bringing an arthropod, preferably insect, into direct contact or in contact with vapors of a composition as described hereinabove.

For the sake of clarity, the arthropod controlling composition according to the present invention can be applied to the air, to the surface of an article, the air in the vicinity of the surface of an article or the surface of a subject by usual methods known in the art such as spraying, applying, wearing or diffusing.

In a particular embodiment, the arthropod controlling composition according to the present invention is applied to the surface of an article, the air in the vicinity of the surface of an article or to the surface of an animal or subject.

In a particular embodiment, the article can be an arthropod control article as described hereinbelow and in particular can be a candle, coil, electric diffuser, wristband, patch, collar, ear tag, clothes, fabrics, papers, biochar, cardboard, cellulosic pads, bed nets, screen, curtains, furniture, paint, walls, ground, spray, aerosol, cream, roll on, wristband, lotion, soap, shampoo, sunscreen, laundry powder, liquid detergent, spray, lotion, powder.

In a particular embodiment, the surface of a subject is the surface of a human or animal subject, preferably the surface is a human subject, i.e. the skin of a human subject.

The present invention also relates to a use of a composition as defined hereinabove to control arthropods, preferably insects.

The present invention also relates to an arthropod control article comprising the arthropod control composition as described hereinabove.

By “arthropod control article” is understood to designate a consumer product which delivers at least an arthropod controlling effect to the surface or space to which it is applied (e.g. skin, hair, textile, or home surface). In other words, an arthropod controlling article according to the invention is a consumer product which comprises a functional formulation, as well as optionally additional benefit agents, corresponding to the desired consumer product, and an arthropod controlling amount of at least one of the substances. For the sake of clarity, said consumer product is a non-edible product.

The nature and type of the constituents of the consumer product do not warrant a more detailed description here, which in any case would not be exhaustive, the skilled person being able to select them on the basis of his general knowledge and according to the nature and the desired effect of said product.

Non-limiting examples of suitable consumer products include a perfume, such as a fine perfume, a splash or eau de parfum, a cologne or a shave or after-shave lotion or a cream or gel; a fabric care product, such as a liquid or solid detergent, a laundry powder, a fabric softener, a liquid or solid scent booster, a fabric refresher, an ironing water, a paper, a bleach, a carpet cleaner, a curtain-care product; a body-care product, such as a hair care product (e.g. a shampoo, a coloring preparation or a hair spray, a color-care product, a hair shaping product), a dental care product, a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner; a candle; a spray, a coil, an electric diffuser, a diffusor, a rubber septum, a wristband, a patch, a collar, an ear tag, clothes, fabrics, papers, a biochar, a cardboard, celluloic pads, bed nets, a screen, curtains, a varnish or a paint, more preferably a candle, a spray, a coil, an electric diffuser, a diffusor, a rubber septum, a wristband, a patch, a collar, an ear tag, clothes, fabrics, papers, a biochar, a cardboard, celluloic pads, bed nets, a screen, curtains, a varnish or a paint.

In a preferred embodiment of the invention, the consumer product is an electric diffuser. In this embodiment of the invention, the substance in the arthropod, preferably insect, control composition is present at certain quantities.

Hence, where the consumer product is an electric diffuser, the substance is selected from the list consisting of: (2E)-3,7-dimethylocta-2,6-dienal; chromen-2-one; 2-methoxy-4-[(E)-prop-1-enyl]phenol; (5R)-2-methyl-5-prop-1-en-2-ylcyclohex-2-en-1-one; (4-methoxyphenyl)methyl acetate; 2-methyl-3-(4-propan-2-ylphenyl)propanal; (4-methoxyphenyl)methanol; 2-phenylethanol; 4-hydroxy-3-methoxybenzaldehyde; Mentha spicata oil; Syzygium aromaticum oil; 5-pentyloxolan-2-one; (1S,6R)-3,7,7-trimethylbicyclo[4.1.0]hept-3-ene; (4-methoxyphenyl)methyl acetate; 2-phenylethyl 2-methylpropanoate.

Moreover where the consumer product is an electric diffuser, then the following amounts are preferred, expressed as a total percentage of the diffuser liquid: (2E)-3,7-dimethylocta-2,6-dienal, 100% or less; chromen-2-one, 33% or less; 2-methoxy-4-[(E)-prop-1-enyl]phenol, 100% or less; (5R)-2-methyl-5-prop-1-en-2-ylcyclohex-2-en-1-one, 16.9% or less; (4-methoxyphenyl)methyl acetate, 20% or less; 2-methyl-3-(4-propan-2-ylphenyl)propanal, 15.6% or less; (4-methoxyphenyl)methanol, 13.8% or less; 2-phenylethanol, 20% or less; 4-hydroxy-3-methoxybenzaldehyde, 20% or less; Mentha spicata oil, 9% or less; Syzygium aromaticum oil, 9% or less; 5-pentyloxolan-2-one, 20% or less; (1S,6R)-3,7,7-trimethylbicyclo[4.1.0]hept-3-ene, 20% or less; (4-methoxyphenyl)methyl acetate, 20% or less; 2-phenylethyl 2-methylpropanoate, 20% or less.

Some of the above-mentioned consumer products may represent an aggressive medium for the members of the substances, so that it may be necessary to protect the latter from premature decomposition, for example by encapsulation or by chemically binding it to another chemical which is suitable to release the invention’s ingredient upon a suitable external stimulus, such as an enzyme, light, heat or a change of pH.

### EXAMPLES

The invention will be described in further detail by way of the following examples.

### 1. Description of the Arthropod Controlling Assays

The arthropod repellency is tested using in vitro and in vivo test methods as described herein.

### 1.1. In Vitro Warm Body Assay for Insects Such as Mosquito

A. aegypti is a model organism for controlling tests and one of the recommended model organisms by the World Health Organization (WHO) as it is a very aggressive anthropophilic mosquito species that shows generally low sensitivity to arthropod controlling compounds.

The controlling effect of the compositions according to the present invention was assessed using an adapted Warm Body assay as defined in Kröber T, Kessler S, Frei J, Bourquin M, Guerin PM. 2010. An in vitro assay for testing mosquito controlling compounds employing a warm body and carbon dioxide as a behavioral activator. J Am Mosq Control Assoc. 26:381-386. In this in-vitro assay the number of mosquito landing on a warm body, simulating an attractive host treated with the tested stimuli, was measured in order to assess the repellence effect.

The published protocol has been adapted in not manually counting the landing mosquitoes but automatically using an automatic counting software, the switch from Anopheles gambiae to Aedes aegypti led to a decrease of mosquitoes’ number placed in the tested cage due to the size difference (i.e. 30 mosquitoes instead of 50) and to an increase of lighting as A. aegypti is a diurnal mosquitoes (i.e. 150 lux instead of 4 lux).

In addition, since the inventors were interested by assessing spatial repellence added or not to a deterrent effect, the substance was applied using two different technics. Whether, as in Kröber et al. (2010), both deterrent and repellent effect were investigated (i.e. similar effect than most body care applications such as lotion) and the substances was applied on a sandblasted glass Petri dish placed on top of the warm body. Whether only the spatial repellence was investigated (i.e. similar effect than most home care applications such as liquid electric dispenser) and the substance was introduced in the cage space thanks to a forced evaporation system as described in Chappuis C J-F, Niclass Y, Vuilleumier C, Starkenmann C. 2015. Quantitative Headspace Analysis of Selected Odorants from Latrines in Africa and India. Environ. Sci. Technol. 49:6134-6140.

As stated in Kröber et al. (2010), selected 10-12 days old hungry females, that had ad libitum access to 10% sugar solution but were not blood-fed, were selected for the assay. For each substance tested, at least three different concentrations were assessed ranging from 0.0016% up to 1% in ethanol for deterrence & repellence assessment and from 0.001 mg/mL up to 100 mg/mL in propylene glycol for spatial repellence.

### 1.2. In Vivo Arm-In-Cage Assay for Insects Such as Mosquito

The Arm in the box method was adapted from the WHO Guidelines for efficacy testing of mosquito repellents for human skin (WHO/CDS/NTD/WHOPES/2009.4). The readiness of 100 hungry female mosquitoes A. aegypti to probe is assessed by inserting an untreated arm into the cage (40×40×40 cm) for 30 seconds (negative control) three times (once at the beginning, once at the fourth hour and once at the eighth hour), to determine probing activity. Then, the product is applied onto the skin of the forearm of a human volunteer (1 mL per 600 cm²) and after 5 minutes, this arm is inserted into the cage and exposed for 3 minutes. The assay take place in temperature (27±2° C.) and humidity (80±10%RH) regulated room on three different volunteers.

### 1.3. In Vitro Warm Plate Assay for Arachnids Such as Ticks

Repellent efficacy of the different substances was assessed against the castor bean tick, Ixodes ricinus L that can transmit both bacterial and viral pathogens. I. ricinus is one of the recommended model organisms mentioned by the Guidance on the European Biological Products Regulation [Vol II, Efficacy - Assessment & Evaluation (Parts B+C), v. 3.0, April 2018]. Observations of repellent efficacy were made on last stage nymphs.

The repellent efficacy was assessed using the protocol of the in-vitro Warm Plate Assay as defined in Kröber T, Bourquin M, Guerin PM. 2013. A standardized in vivo and in vitro test method for evaluating tick repellents. Pestic. Biochem. Phys. 107(2):160-168.

### 2. Evaluation of the Arthropod Controlling Effect of the Present Invention

### 2.1. Results of in Vitro Warm Body Assay for Insects Such as Mosquito

### 2.1.1. Results of in Vitro Assay for Deterrence and Spatial Repellence

On the sandblasted glass Petri dish covering the warm body (28.3 cm²), 100 µL of the substances diluted at different concentrations in ethanol was applied. The number of mosquito landing on the warm body was counted for each stimuli, N,N-diethyl-3-methylbenzamide (DEET) and ethyl 3-[acetyl(butyl)amino]propanoate (IR3535) being used as benchmarks and pure ethanol as control.

All substances tested elicited a clear decrease of landings linked to the increase of the applied dose. At a concentration of 0.04%, two substances (chromen-2-one & 4-hydroxy-3-methoxybenzaldehyde) obtained similar number of landings to DEET, with less than 10 landings in total. All additional substances tested were, at this concentration of 0.04%, as efficient as the second benchmark IR3535 with ~10 landings per minute (Table 1). At a higher concentration of 1%, all seven substances tested demonstrated a number of mosquito landing similar to DEET and lower than IR3535 with less than 2 mosquito landing per minute (Table 1).

TABLE 1

number of mosquitoes landing within 2 minutes on the warm body treated with different substances at different concentrations. Test with ethanol only (control) elicited 59.3±6.9 landings of mosquito

| | | | |
|---|---|---|---|
| | 0.0016% | 0.04% | 1% |
| N,N-diethyl-3-methylbenzamide | 44.9 | 5.1 | 2.6 |
| ethyl 3-[acetyl(butyl)amino]propanoate | 70.0 | 20.7 | 7.7 |
| 2-methyl-3-(4-propan-2-ylphenyl)propanal | 49.0 | 20.0 | 1.0 |
| (4-methoxyphenyl)methanol | 72.0 | 16.0 | 0.0 |
| chromen-2-one | 118.0 | 0.0 | 1.0 |
| 5-pentyloxolan-2-one | 76.0 | 16.0 | 1.0 |
| (2E)-3,7-dimethylocta-2,6-dienal | 89.0 | 16.0 | 2.0 |
| (5R)-2-methyl-5-prop-1-en-2-ylcyclohex-2-en-1-one | 24.0 | 16.0 | 3.0 |
| 4-hydroxy-3-methoxybenzaldehyde | 19.0 | 9.0 | 3.0 |

All substances tested elicited a clear decrease of landings correlated with the increase of the applied dose. At a concentration of 0.04%, two substances s (4-ethenyl-2-methoxyphenol & (3Z)-3-butylidene-2-benzofuran-1-one) obtained similar number of landings to DEET with less than 10 landings in total. Most of the additional substances tested were, at this concentration of 0.04%, as efficient as the second benchmark IR3535 with ~10 landings per minute (Table 2). At a higher concentration of 1%, six of the eight substances tested demonstrated a number of mosquito landing similar to DEET and lower than IR3535 with less than 3 mosquito landing per minutes (Table 2).

TABLE 2

number of mosquitoes landing within 2 minutes on the warm body treated with different substances at different concentrations. Test with ethanol only (control) elicited 67.1±9.1 landings of mosquito

| | | | |
|---|---|---|---|
| | 0.0016% | 0.04% | 1% |
| N,N-diethyl-3-methylbenzamide | 44.9 | 5.1 | 2.6 |
| ethyl 3-[acetyl(butyl)amino]propanoate | 70.0 | 20.7 | 7.7 |
| 4-ethenyl-2-methoxyphenol | 36.0 | 5.0 | 0.0 |
| Nigella damascena oil | 53.0 | 11.0 | 1.0 |
| (E)-1-(2,6,6-trimethylcyclohex-2-en-1-yl)but-2-en-1-one | 70.0 | 34.0 | 9.0 |
| Labdanum extract | 96.0 | 33.0 | 2.0 |
| Cognac oil green | 53.0 | 17.0 | 2.0 |
| (3Z)-3-butylidene-2-benzofuran-1-one | 54.0 | 9.0 | 1.0 |
| 6-hexyloxan-2-one | 74.0 | 13.0 | 5.0 |
| 5-methyl-2-propan-2-ylcyclohexyl] acetate | 22.0 | 16.0 | 10.0 |

All substances tested elicited a clear decrease of landings linked to the increase of the applied dose. At a concentration of 0.04%, three substances (methyl 2-(3-oxo-2-pent-2-enylcyclopentyl)acetate, 6-pentyloxan-2-one & 3,4,4a,5,6,7,8,8a-octahydrochromen-2-one) obtained similar number of landings to DEET with less than 10 landings in total. At this same concentration of 0.04%, three other stimuli (2-methoxy-4-[(E)-prop-1-enyl]phenol, (4R)-4-(2-methoxypropan-2-yl)-1-methylcyclohexene) and Syzygium aromaticum oil obtained similar number of landings to IR3535 with ~10 landings per minute (Table 3). At higher concentration of 1%, nine of the twelve substances tested demonstrated a number of mosquito landing similar to DEET and lower than IR3535 with less than 3 mosquito landing per minutes. 3,7,7-trimethylbicyclo[4.1.0]hept-3-ene & (4-methoxyphenyl)methyl acetate obtained similar repellent results to IR3535 at this concentration of 1%, while 1% of Mentha spicata oil managed to decrease the number of mosquitoes landings by 80% (Table 3).

TABLE 3

number of mosquitoes landing within 2 minutes on the warm body treated with different substances at different concentrations. Test with ethanol only (control) elicited 67.0±8.8 landings of mosquito

| | | | |
|---|---|---|---|
| | 0.0016% | 0.04% | 1% |
| N,N-diethyl-3-methylbenzamide | 44.9 | 5.1 | 2.6 |
| ethyl 3-[acetyl(butyl)amino]propanoate | 70.0 | 20.7 | 7.7 |
| methyl 2-(3-oxo-2-pent-2-enylcyclopentyl)acetate | 54.0 | 2.0 | 1.0 |
| 6-pentyloxan-2-one | 90.0 | 7.0 | 2.0 |
| 3,4,4a,5,6,7,8,8a-octahydrochromen-2-one | 45.0 | 7.0 | 0.0 |
| 2-methoxy-4-[(E)-prop-1-enyl]phenol | 57.0 | 14.0 | 0.0 |
| (4R)-4-(2-methoxypropan-2-yl)-1-methylcyclohexene | 52.0 | 18.0 | 3.0 |
| (4-methoxyphenyl)methyl acetate | 59.0 | 27.0 | 9.0 |
| 2-phenylethanol | 72.0 | 46.0 | 0.0 |
| (1S,6R)-3,7,7-trimethylbicyclo[4.1.0]hept-3-ene | 59.0 | 55.0 | 8.0 |
| 2-phenylethyl 2-methylpropanoate | 90.0 | 55.0 | 2.0 |
| Syzygium aromaticum oil | 32.0 | 11.0 | 0.0 |
| Mentha spicata oil | 58.0 | 78.0 | 13.0 |
| Mentha piperita oil | 48.0 | 37.0 | 0.0 |

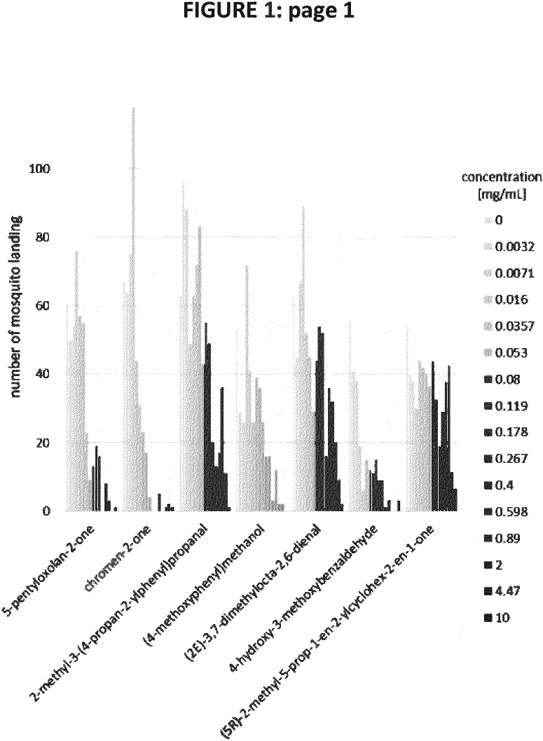

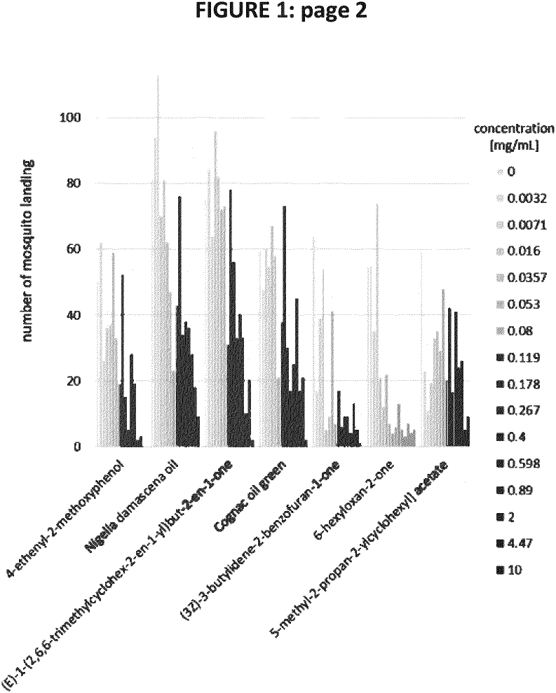

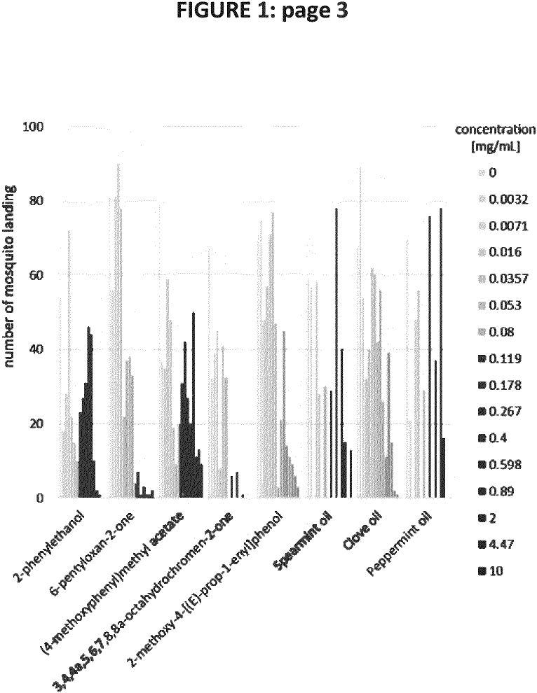

Similar were found by testing the above-mentioned stimuli with additional concentrations as can be seen from the data in FIG. 1.

### 2.1.2. Results of in vitro Assay for Spatial Repellence

The forced evaporation system allows to fill the volume of the cage with stimuli tested, (2-methyl-4-oxo-3-prop-2-ynylcyclopent-2-en-1-yl) 2,2-dimethyl-3-(2-methylprop-1-enyl)cyclopropane-1-carboxylate (prallethrin) and N,N-diethyl-3-methylbenzamide (DEET) being used as benchmarks. In order to limit potential dead volumes in the cage, five flushes of the cage were made before starting the 2 minutes test during which the number of mosquito landings was counted.

Like the two benchmarks, all substances efficacy to avoid landing on the warm body increased as a dose response, except 3,7-dimethylocta-2,6-dienal that provided a ~67% decrease of landings identical at all concentrations tested. The ⅔ decrease of landing was observed in most substances tested at the dose of 0.0017 µg/µL of air. For all substances tested the number of landings decreased to reach less than five landings per minutes at doses 1.7 and/or 17 µg/L of air (Table 4).

TABLE 4

number of mosquitoes landing within 2 minutes on the warm body while the air in the cage was saturated with a stimulus. The mean number of landing without substance in the cage was 54.7±3.2. n=1-4 replicates per concentration

| | 0.0017 µg/L air | 0.017 µg/L air | 0.17 µg/L air | 1.7 µg/L air | 17 µg/L air |
|---|---|---|---|---|---|
| prallethrin | 77.0 | 66.0 | 15.5 | 3.0 | 1.0 |
| N,N-diethyl-3-methylbenzamide | 35.0 | 28.0 | 24.0 | 1.5 | 3.0 |
| 2-methyl-3-(4-propan-2-ylphenyl)propanal | 26.3 | 21.3 | 7.8 | 5.0 | 7.5 |
| (4-methoxyphenyl)methanol | 21.5 | 27.3 | 8.5 | 4.0 | 20.0 |
| 5-pentyloxolan-2-one | 14.7 | 23.0 | 15.3 | 7.7 | 2.0 |
| chromen-2-one | 36.0 | 3.0 | 10.0 | 1.0 | 0.0 |
| (2E)-3,7-dimethylocta-2,6-dienal | 25.3 | 15.7 | 15.3 | 14.7 | 23.0 |
| 4-hydroxy-3-methoxybenzaldehyde | 17.0 | 9.0 | 1.5 | 3.5 | 1.0 |
| (5R)-2-methyl-5-prop-1-en-2-ylcyclohex-2-en-1-one | 20.0 | 27.0 | 2.0 | 2.0 | 2.0 |

For all substance, there was a decrease in the number of landings linked to the increase of quantity of substance present in the air of the cage. Four substances (Cognac oil green, 4-ethenyl-2-methoxyphenol, (E)-1-(2,6,6-trimethylcyclohex-2-en-1-yl)but-2-en-1-one & 6-hexyloxan-2-one) managed to obtain less than five landings in 2 minutes at a low dose of 0.17 µg/L of air. All other substances reached a level of 90% of repulsion at doses of 1.7 and/or 17 µg/L in air with the exception of 3-butylidene-2-benzofuran-1-one. (E)-1-(2,6,6-trimethylcyclohex-2-en-1-yl)but-2-en-1-one is particularly efficient, as there was more than 80% repulsion at all five tested concentrations (Table 5).

TABLE 5

number of mosquitoes landing within 2 minutes on the warm body while the air in the cage was saturated with a stimulus. The mean number of landing without substance in the cage was 56.0±3.6. n=1-4 replicates per concentration. n.d. indicates that no measure was carried

| | 0.0017 µg/L air | 0.017 µg/L air | 0.17 µg/L air | 1.7 µg/L air | 17 µg/L air |
|---|---|---|---|---|---|
| Prallethrin | 77.0 | 66.0 | 15.5 | 3.0 | 1.0 |
| N,N-diethyl-3-methylbenzamide | 35.0 | 28.0 | 24.0 | 1.5 | 3.0 |
| Labdanum extract | 60.0 | 61.0 | 33.0 | 3.0 | 19.0 |
| Cognac oil green | 23.0 | 29.0 | 1.0 | 2.0 | 3.0 |
| 4-ethenyl-2-methoxyphenol | 11.0 | 15.0 | 0.0 | 3.0 | 2.0 |
| (3Z)-3-butylidene-2-benzofuran-1-one | 59.0 | 31.5 | 17.0 | 11.0 | 37.0 |
| 5-methyl-2-propan-2-ylcyclohexyl] acetate | 31.5 | 24.0 | 17.5 | 9.0 | 25.0 |
| 1-(2,6,6-trimethylcyclohex-2-en-1-yl)but-2-en-1-one | 6.0 | 11.0 | 4.0 | 4.0 | 1.0 |
| 6-hexyloxan-2-one | 31.0 | 28.0 | 4.0 | n.d. | 5.0 |
| Nigella damascena oil | 46.5 | 37.5 | 10.7 | 7.5 | 0.0 |

For all substances, we observed a decrease of the number of landing linked to the increase of quantity of substance present in the air of the cage. However, this decrease was very poor for some substances such as 2-phenylethanol with 41% of landing decrease at the highest dose tested. In contrast, some substances worked very well, with more than 90% of landing decrease at dose of 0.017 µg/µL. (Table 6)

TABLE 6

number of mosquitoes landing within 2 minutes on the warm body while the air in the cage was saturated with a stimulus. The mean number of landing without substance in the cage was 54.1±4.9. n=1-4 replicates per concentration. n.d. indicates that no measure was carried

| | 0.0017 µg/L air | 0.017 µg/L air | 0.17 µg/L air | 1.7 µg/L air | 17 µg/L air |
|---|---|---|---|---|---|
| prallethrin | 77.0 | 66.0 | 15.5 | 3.0 | 1.0 |
| N,N-diethyl-3-methylbenzamide | 35.0 | 28.0 | 24.0 | 1.5 | 3.0 |
| 2-phenylethanol | 12.0 | 30.0 | 25.0 | 51.0 | 30.0 |
| 6-pentyloxan-2-one | 43.0 | 4.0 | 28.0 | 0.5 | 0.0 |
| (4-methoxyphenyl)methyl acetate | 18.0 | 26.5 | 34.5 | 28.0 | n.d. |
| methyl 2-(3-oxo-2-pent-2-enylcyclopentyl)acetate | n.d. | 10.0 | 4.0 | 15.0 | n.d. |
| 2-methoxy-4-[(E)-prop-1-enyl]phenol | n.d. | 12.0 | 4.0 | 11.0 | n.d. |
| Syzygium aromaticum oil | 13.0 | 31.0 | 1.5 | 0.0 | 5.0 |
| Mentha spicata oil | 23.0 | 24.0 | 1.0 | 2.0 | 9.0 |
| Mentha piperita oil | n.d. | 11.0 | 4.0 | 3.0 | n.d. |

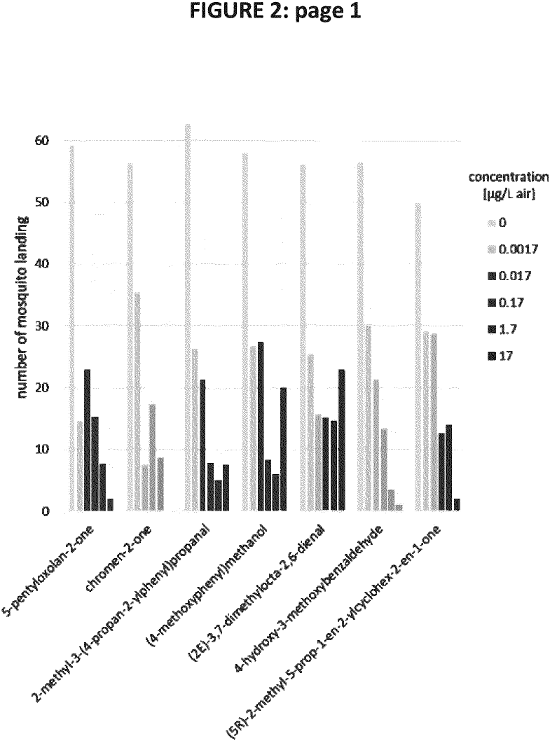

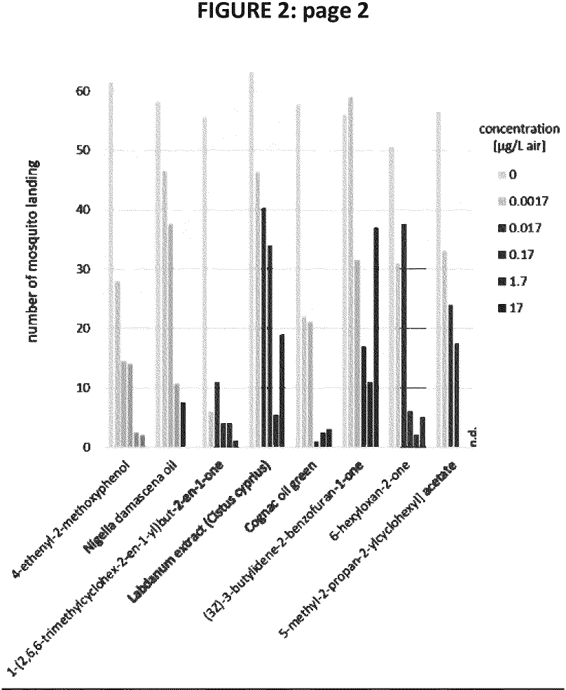

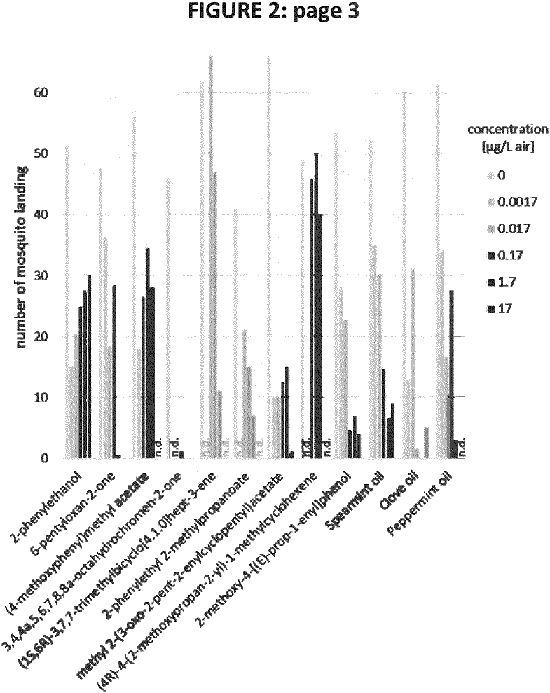

These dose responses can be clearly visualized from the data presented in FIG. 2.

### 2.2. Results of in vivo Arm-In-Cage Assay for Insects Such as Mosquito

Arm-in-cage test is a compulsory assay for most biocide registrations to assess the efficacy of a substance. It is a very challenging test as the treated arm of a volunteer is being placed in a space containing a density of 3125 blood hungry mosquitoes / m³ of air. Application of 20% of 4-hydroxy-3-methoxybenzaldehyde on three different volunteers arm skin (600 cm²), nevertheless managed to decrease the percentage of mosquito landing on the arm by 82±5.2%, 78±2.6%, 66±8.0%, 61±6% and 63±2% at the different testing times post-applications (5 min, 1 h, 2 h, 3 h and 4 h respectively).

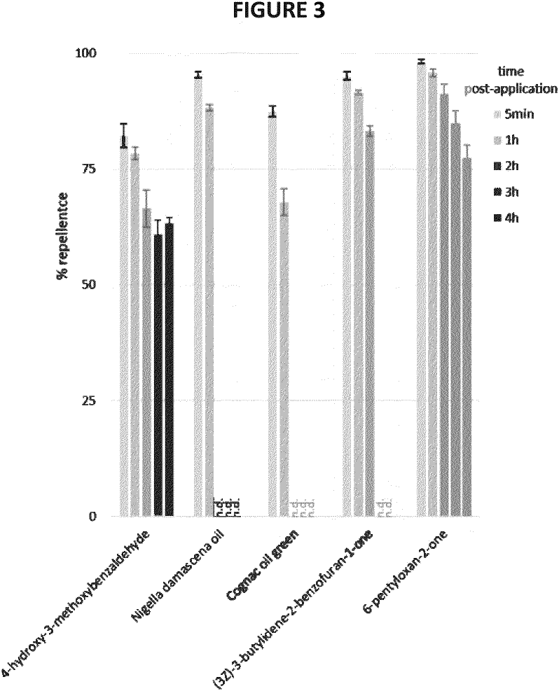

Similarly, other compounds demonstrated relevant repellent effect, especially 6-pentyloxan-2-one that managed to stay above 75% of repellence for more than 4h (data provided in FIG. 3).

### 2.3. Results of in vitro Warm Plate Assay for Arachnids Such as Tick

If the tick exhibits the preset negative geotactic response in the in-vitro assay, it demonstrates that the tested substance does not prevent the tick from climbing up in search of a suitable attachment site for a blood meal in nature. The mean percentage of ticks affected by three concentrations of substances diluted in ethanol was therefore measured on 12-24 ticks, using ethyl 3-[acetyl(butyl)amino]propanoate (IR3535) as benchmark and pure ethanol as control. A clear biological dose response was measured, i.e. the number of ticks affected by all substances increased with the quantity of substances applied. The most efficient tested substances were chromen-2-one and 5-pentyloxolan-2-one both of which provided a response similar to the benchmark IR3535 at the three concentrations tested. Even the least efficient substances managed, at the highest concentration of 1% (25 µg/cm²) tested, to decrease the number of ticks climbing in search of a feeding site to more than 50% (Table 7).

TABLE 7

mean percentage of ticks affected by the substances diluted in ethanol. During test with ethanol (control), only 1.0±1.2% of ticks were affected

| | 1 µg/cm² | 5 µg/cm² | 25 µg/cm² |
|---|---|---|---|
| ethyl 3-[acetyl(butyl)amino]propanoate | 33% | 67% | 91% |
| chromen-2-one | 27% | 71% | 80% |
| 2-methyl-3-(4-propan-2-ylphenyl)propanal | 7% | 46% | 85% |
| (4-methoxyphenyl)methanol | 6% | 8% | 58% |
| (2E)-3,7-dimethylocta-2,6-dienal | 15% | 0% | 58% |
| 5-pentyloxolan-2-one | 31% | 83% | 100% |
| 4-hydroxy-3-methoxybenzaldehyde | 14% | 44% | 73% |
| (5R)-2-methyl-5-prop-1-en-2-ylcyclohex-2-en-1-one | 50% | 17% | 57% |

3-butylidene-2-benzofuran-1-one has a greater tick repellency, than the benchmark compound IR3535, i.e. at the lowest dose tested (0.04%), more than ¾ of the ticks were affected while at the intermediate dose tested (0.2%) all tested ticks were affected and did not search for a feeding site. All other substances demonstrated a dose response efficacy to repel the ticks, reaching at minima 50% repulsion when tested at 1%. 4-ethenyl-2-methoxyphenol also managed to avoid all tested ticks at the highest concentration of 1% (25 µg/cm²) to climb-up in search of a feeding site (Table 8).

TABLE 8

mean percentage of ticks affected by the substance diluted in ethanol. During test with ethanol (control), only 4.3±2.1% of ticks were affected

| | 1 µg/cm² | 5 µg/cm² | 25 µg/cm² |
|---|---|---|---|
| ethyl 3-[acetyl(butyl)amino]propanoate | 33% | 67% | 91% |
| 4-ethenyl-2-methoxyphenol | 17% | 46% | 100% |
| Labdanum extract | 6% | 41% | 53% |
| Cognac oil green | 7% | 0% | 62% |
| (3Z)-3-butylidene-2-benzofuran-1-one | 77% | 100% | 100% |
| 1-(2,6,6-trimethylcyclohex-2-en-1-yl)but-2-en-1-one | 13% | 6% | 83% |

Both tested substances managed to decrease the percentage of ticks climbing-up in search of a feeding site. This percentage of repulsion was similar to the benchmark IR3535 at the highest doses tested of 0.2% and 1% (5 & 25 µg/cm² respectively ; Table 9).

TABLE 9

mean percentage of ticks affected by the substances diluted in ethanol. During test with ethanol (control), only 0.6±0.5% of ticks were affected

| | 0.04 µg/cm² | 1 µg/cm² | 5 µg/cm² | 25 µg/cm² |
|---|---|---|---|---|
| ethyl 3-[acetyl(butyl)amino]propanoate | 13% | 33% | 67% | 91% |
| 3,4,4a,5,6,7,8,8a-octahydrochromen-2-one | 33% | 17% | n.d. | 92% |
| 2-methoxy-4-[(E)-prop-1-enyl]phenol | n.d. | 8% | 67% | 83% |

1. An arthropod, control composition comprising one or more substances, wherein the substance is selected from the group consisting of: (E)-1-(2,6,6-trimethylcyclohex-2-en-1-yl)but-2-en-1-one, (3Z)-3-butylidene-2-benzofuran-1-one, 4-ethenyl-2-methoxyphenol, Cognac oil green, Labdanum extract (Cistus spp.), 5-pentyloxolan-2-one, chromen-2-one, (2E)-3,7-dimethylocta-2,6-dienal, 4-hydroxy-3-methoxybenzaldehyde, (5R)-2-methyl-5-prop-1-en-2-ylcyclohex-2-en-1-one, Mentha spicata oil, 6-hexyloxan-2-one, 5-methyl-2-propan-2-ylcyclohexyl] acetate, Nigella damascena oil, 2-phenylethanol, 6-pentyloxan-2-one, (4-methoxyphenyl)methyl acetate, Syzygium aromaticum oil, 3,4,4a,5,6,7,8,8a-octahydrochromen-2-one, (1S,6R)-3,7,7-trimethylbicyclo[4.1.0]hept-3-ene, 2-phenylethyl 2-methylpropanoate, methyl 2-(3-oxo-2-pent-2-enylcyclopentyl)acetate, (4R)-4-(2-methoxypropan-2-yl)-1-methylcyclohexene, Mentha piperita oil, 2-methoxy-4-[(E)-prop-1-enyl]phenol, 2-methyl-3-(4-propan-2-ylphenyl)propanal and (4-methoxyphenyl)methanol.

2. The arthropod control composition according to claim 1, wherein the arthropod is an insect.

3. The arthropod control composition according to claim 1, wherein the substance is used for repellence and is selected from Labdanum extract, Cognac oil green, (3Z)-3-butylidene-2-benzofuran-1-one, 2-methyl-3-(4-propan-2-ylphenyl)propanal, methyl 2-(3-oxo-2-pent-2-enylcyclopentyl)acetate, Nigella damascena oil, (2E)-3,7-dimethylocta-2,6-dienal, 5-pentyloxolan-2-one, Syzygium aromaticum oil, 4-ethenyl-2-methoxyphenol, 4-hydroxy-3-methoxybenzaldehyde, 6-pentyloxan-2-one, (1S,6R)-3,7,7-trimethylbicyclo[4.1.0]hept-3-ene, chromen-2-one, 6-hexyloxan-2-one, 2-methoxy-4-[(E)-prop-1-enyl]phenol, 5-methyl-2-propan-2-ylcyclohexyl] acetate, or (4-methoxyphenyl)methanol.

4. The arthropod control composition according to claim 1 wherein the substance is used for spatial repellence and is selected from Cognac oil green, (E)-1-(2,6,6-trimethylcyclohex-2-en-1-yl)but-2-en-1-one, 2-methyl-3-(4-propan-2-ylphenyl)propanal, 4-ethenyl-2-methoxyphenol, methyl 2-(3-oxo-2-pent-2-enylcyclopentyl)acetate, 6-hexyloxan-2-one, chromen-2-one, 6-pentyloxan-2-one, Mentha piperita oil, 4-hydroxy-3-methoxybenzaldehyde, Syzygium aromaticum oil, 5-pentyloxolan-2-one, 2-phenylethanol, Nigella damascena oil, Mentha spicata oil, (5R)-2-methyl-5-prop-1-en-2-ylcyclohex-2-en-1-one, 2-methoxy-4-[(E)-prop-1-enyl]phenol, (4-methoxyphenyl)methanol, (4R)-4-(2-methoxypropan-2-yl)-1-methylcyclohexene, 2-methoxy-4-[(E)-prop-1-enyl]phenol, or (4-methoxyphenyl)methyl acetate.

5. The arthropod control composition according to claim 1, wherein the arthropod is an arachnid.

6. The arthropod control composition according to claim 1 wherein the substance is used for deterrence and is selected from (E)-1-(2,6,6-trimethylcyclohex-2-en-1-yl)but-2-en-1-one, 4-ethenyl-2-methoxyphenol, (3Z)-3-butylidene-2-benzofuran-1-one, 5-pentyloxolan-2-one, chromen-2-one, 2-methoxy-4-[(E)-prop-1-enyl]phenol, or 2-methyl-3-(4-propan-2-ylphenyl)propanal.

7. The arthropod control composition according to claim 1, wherein the arthropod control composition comprises the substance in an amount of 0.01 to 90 wt.%, based on the total weight of the composition.

8. The arthropod control composition according to claim 1, comprising at least two substances.

9. The arthropod control composition according to claim 1, further comprising an arthropod control co-ingredient selected from the group consisting of N,N-diethyl-3-methylbenzamide (DEET), ethyl butylacetylaminopropionate (IR3535); para-menthan-3,8-diol (PMD), 1-(1-methylpropoxycarbonyl)-2-(2-hydroxaethyl)piperidin (Icaridin); Cedarwood oil (China), Cedarwood oil (Texas), Cedarwood oil (Virginia), Cinnamon oil, Citronella oil, Cornmint oil, Cymbopogon winterianus oil fractionated hydrated cyclized, decanoic acid, Eucalyptus citriodora oil, Eucalyptus citriodora oil hydrated cyclized, eugenol, Garlic oil, geraniol, Geranium oil, Lavender, Lavandula hybrida ext., Lavandin oil, Lemon oil, Lemongrass oil, Margosa extract, Metofluthrin, mixture of cis- and trans-p-menthane-3,8 diol, N,N-diethyl-meta-toluamide, nonanoic acid, Rosemary oil, Thyme oil, Wintergreen oil, 2,3,4,5-bis(butyl-2-ene)tetrahydrofurfural (MGK Repellent 11), cineole, cinnamaldehyde, citronellal, citronellol,, coumarin, dibutyl phthalate, diethyl phthalate, dimethyl anthranilate, dimethyl phthalate, ethyl vanillin, Eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, Lime oil, limonene, linalool, methyl anthranilate, Mint oil, myrcene, Neem oil, sabinene, β-caryophyllene, (1H-indol-2-yl)acetic acid, anethole, Anise oil, Basil oil, Bay oil, camphor, ethyl salicylate, Evergreen oils (pine oil), (1,3,4,5,6,7-hexahydro-1,3-dioxo-2H-isoindol-2-yl)methyl (1R-trans)-2,2-dimethyl-3-(2-methylprop-1-enyl)cyclopropanecarboxylate (d-Tetramethrin), (RS)-3-Allyl-2-methyl-4-oxocyclopent-2-enyl-(1R,3R;1R,3S)-2,2-dimethyl-3-(2- methylprop-1-enyl)-cyclopropanecarboxy late (mixture of 4 isomers 1R trans, 1R:1R trans, 1S: 1R cis, 1R: 1R cis, 1S 4:4:1:1) (d-Allethrin), (RS)-α-cyano-3phenoxybenzyl-(1RS)-cis, trans-3-(2,2-dichlorovinyl)-2,2-dimethylcyclopropanecarboxylate (Cypermethrin), 2-methyl-4-oxo-3-(prop-2-ynyl)cyclopent-2-en-1-yl 2,2-dimethyl-3-(2-methylprop-1-enyl)cyclopropanecarboxylate (Prallethrin), Acetamiprid, Azadirachtin, Bendiocarb, Bifenthrin, boric acid, Chlorpyrifos, Deltamethrin, Diazinon, Dichlorvos, eugenol, Fipronil, Imidacloprid, linalool, Malathion, Maltodextrin, Metofluthrin, Nicotine, Permethrin, Pyrethrins and Pyrethroids, Rotenone, silicium dioxide (Kieselguhr), S-Methoprene, Spinosad (Spinosyn A), Spinosyn D, Tetramethrin, Transfluthrin and mixtures thereof.

10. A method for arthropod, control which comprises bringing an insect into direct contact or in contact with vapors of a composition as defined in claim 1.

11. The method according to claim 10, wherein the arthropod is an insect.

12. An arthropod, control article comprising the arthropod, control composition according to claim 1.

13. The arthropod control article according to claim 12, wherein the article is a consumer product.

14. The arthropod control article according to claim 12 wherein the article is a fabric care product; a body-care product; a cosmetic preparation; a skin-care product; an air care product; or a home care product; a leather care product; a car care product; a candle; a spray; a coil, an electric diffuser, a diffusor, a rubber septum, a wristband, a patch, a collar, an ear tag, clothes, fabrics, papers, a biochar, a cardboard, cellulosic pads, bed nets, a screen, curtains, a varnish or a paint.

15. The arthropod control article according to claim 14, wherein the article is an electric diffuser.

16. The arthropod control composition according to claim 2, wherein the arthropod is a mosquito.

17. The arthropod control composition according to claim 5, wherein the arthropod is a tick.

18. The arthropod control composition according to claim 7, wherein the arthropod control composition comprises the substance in an amount of 0.2 to 30 wt.%, based on the total weight of the composition.

14. The arthropod control article according to claim 14, wherein the article is a liquid or solid detergent, a fabric softener, a liquid or solid scent booster, a fabric refresher, an ironing water, a fabric care paper, a bleach, a carpet cleaner, a curtain-care product, a hair care product, a shampoo, a hair coloring preparation or a hair spray, a hair color-care product, a hair shaping product, a dental care product, a disinfectant, an intimate care product, a skin cream or lotion, a vanishing cream, a deodorant or antiperspirant, a hair remover, a tanning or sun or after sun product, a nail product, a skin cleansing, a makeup, a soap, a shower or bath mousse, a bath oil, a bath gel, a hygiene product, a foot/hand care product, an air freshener, a “ready to use” powdered air freshener which can be used in the home space and/or in a public space, a mold remover, a furnisher care product, a wipe, a dish detergent or a hard-surface detergent, a polish, a wax or a plastic cleaner.
